(19) United States
(12) Patent Application Publication
(10) Pub. No.: US 2026/0215829 A1
(43) Pub. Date: Jul. 30, 2026

(54) ORTHOPEDIC PLATING SYSTEM

(71) Applicant: Paragon 28, Inc., Englewood, CO (US)

(72) Inventors: Kerry MCHUGH, Denver, CO (US); Kaitlin KARAS, Denver, CO (US); Clayton BETTIN, Memphis, TN (US); John KWON, Weston, MA (US); Eric MOGHADAMIAN, Versailles, KY (US); Aaron PERDUE, South Lyon, MI (US); Spanky RAYMOND, Uniontown, OH (US)

(73) Assignee: Paragon 28, Inc., Englewood, CO (US)

(21) Appl. No.: 19/462,609

(22) Filed: Jan. 28, 2026

Related U.S. Application Data
(60) Provisional application No. 63/751,592, filed on Jan. 30, 2025.
(60) Provisional application No. 63/854,060, filed on Jul. 30, 2025.

(51) Int. Cl. A61B 17/80; A61B 17/17

(52) U.S. Cl. CPC A61B 17/8061; A61B 17/1728

(57) ABSTRACT

An orthopedic plate includes an upper surface positioned opposite the plate from a lower surface, and at least one through hole extending between the upper and lower surfaces so as to establish fluid communication therebetween. The plate also includes a body portion, a base portion integral with and extending from the body portion, and an extension integral with and extending from the body portion in a direction substantially opposite that of the base portion. Also disclosed is an orthopedic plating system. The system includes an orthopedic plate that has a body portion, a base portion integral with the body portion and an extension integral with the body portion and the base portion. The system further includes a drill guide that is releasably couplable with the body portion of the orthopedic plate.

### CROSS REFERENCE TO RELATED APPLICATION

This application claims priority benefit under 35 U.S.C. § 119(e) of U.S. Provisional Application No. 63/751,592 filed Jan. 30, 2025, entitled Orthopedic Plating System and U.S. Provisional Application No. 63/854,060 filed Jul. 30, 2025, entitled Orthopedic Plating System, which are both incorporated herein by reference in their entirety.

### TECHNICAL FIELD

The present disclosure relates to plates to be implemented in surgical procedures. The present disclosure relates to a podiatric and orthopedic plates to be implemented in procedures of the foot and ankle. More specifically, but not exclusively, the present disclosure relates to an orthopedic plating to be implemented in procedures to address fractures and deformities of the tibia.

### BACKGROUND OF THE INVENTION

Many currently available orthopedic plates do not completely address the needs of patients. Additionally, many currently available orthopedic plates fail to account for properties of foot and ankle anatomy and accordingly can decrease favorability of the outcome for the patient.

### SUMMARY OF INVENTION

The present disclosure is directed toward orthopedic plates to be implemented in procedures of the foot and ankle.

A first aspect of the present disclosure is an orthopedic plate includes an upper surface positioned opposite the plate from a lower surface, and at least one through hole extending between the upper and lower surfaces so as to establish fluid communication therebetween. The plate also includes a body portion, a base portion integral with and extending from the body portion, and an extension integral with and extending from the body portion in a direction substantially opposite that of the base portion.

According to the first aspect of the present disclosure, the extension has a lesser width than the body portion or the base portion.

According to the first aspect of the present disclosure, wherein the upper surface of the base portion is positioned at a substantially orthogonal angle relative to the front surface of the body portion.

According to the first aspect of the present disclosure, the upper surface of the base portion is positioned at an oblique angle relative to at least a portion of the front surface of the extension.

According to the first aspect of the present disclosure, the upper surface of the extension is substantially curved.

According to the first aspect of the present disclosure, the upper surface of the extension at a terminal end of the extension is positioned in a plane angled substantially at about 45-degrees relative to the upper surface of the base portion.

According to the first aspect of the present disclosure, the body portion includes a substantially convex geometry on the upper surface and a substantially concave geometry on the lower surface.

According to the first aspect of the present disclosure, the body portion includes at least one compression slot extending from the upper surface through to the lower surface.

A second aspect of the present disclosure is directed to an orthopedic plating system. The system includes an orthopedic plate having a body portion, a base portion integral with the body portion, and an extension integral with the body portion and the base portion. The system also includes a drill guide.

According to the second aspect of the present disclosure, the drill guide is releasably couplable with the body portion of the orthopedic plate.

According to the second aspect of the present disclosure, the orthopedic plate includes upper and lower surfaces substantially opposite the plate from one another and extending along the base portion, the body portion, and the extension.

According to the second aspect of the present disclosure, at least a portion of the upper surface has a substantially convex geometry and at least a portion of the lower surface has a substantially concave geometry.

According to the second aspect of the present disclosure, the drill guide includes an upper surface substantially opposite the drill guide from a lower surface, and wherein the upper surface includes a substantially convex geometry and the lower surface has a substantially concave geometry.

According to the second aspect of the present disclosure, the body portion includes at least one through hole extending through from the upper surface through to the lower surface, and also at least one coupling feature.

According to the second aspect of the present disclosure, the drill guide includes at least one through hole extending from the upper surface through to the lower surface, and also at least one coupling feature having a geometry complementary to that of the coupling feature of the body portion of the orthopedic plate.

According to the second aspect of the present disclosure, the at least one through hole of the drill guide is coaxial with the at least one through hole of the body portion of the orthopedic plate when the coupling feature of the drill guide is engaged with the coupling feature of the body portion.

According to the second aspect of the present disclosure, the body portion of the orthopedic plate includes at least two through holes extending from the upper surface through to the lower surface and the drill guide includes at least two through holes extending from the upper surface through to the lower surface, wherein each of the at least two through holes of the drill guide and the body portion are coaxial when the coupling features of the drill guide and the body portion are engaged with one another.

According to the second aspect of the present disclosure, the axes of the coaxial through holes of the drill guide and the body portion form a substantially oblique angle with one another.

According to the second aspect of the present disclosure, the axes of the coaxial through holes of the drill guide and the body portion are substantially parallel to one another.

According to the second aspect of the present disclosure, the coupling feature of the body portion includes at least one of a through hole or a depression, and the coupling feature of the drill guide includes a protrusion.

### BRIEF DESCRIPTION OF THE DRAWINGS

The accompanying drawings, which are incorporated in and constitute a part of the specification, illustrate embodiments of the inventions and together with the detailed description herein, serve to explain the principles of the inventions. It is emphasized that, in accordance with the standard practice in the industry, various features may or may not be drawn to scale. In fact, the dimensions of the various features may be arbitrarily increased or reduced for clarity of discussion. The drawings are only for purposes of illustrating embodiments of inventions of the disclosure and are not to be construed as limiting the inventions.

FIG. 1 is a front, left side perspective view of an exemplary orthopedic plate, in accordance with the present disclosure;

FIG. 2 is a rear, right side perspective view of the exemplary orthopedic plate of FIG. 1, in accordance with the present disclosure;

FIG. 3 is a front view of the exemplary orthopedic plate of FIG. 1, in accordance with the present disclosure;

FIG. 4 is a rear view of the exemplary orthopedic plate of FIG. 1, in accordance with the present disclosure;

FIG. 5 is a left side view of the exemplary orthopedic plate of FIG. 1, in accordance with the present disclosure;

FIG. 6 is a right side view of the exemplary orthopedic plate of FIG. 1, in accordance with the present disclosure;

FIG. 7 is a top view of the exemplary orthopedic plate of FIG. 1, in accordance with the present disclosure;

FIG. 8 is a bottom view of the exemplary orthopedic plate of FIG. 1, in accordance with the present disclosure;

FIG. 9 is a front, left side perspective view of an exemplary orthopedic plate, in accordance with the present disclosure;

FIG. 10 is a rear, right side perspective view of the exemplary orthopedic plate of FIG. 9, in accordance with the present disclosure;

FIG. 11 is a front view of the exemplary orthopedic plate of FIG. 9, in accordance with the present disclosure;

FIG. 12 is a rear view of the exemplary orthopedic plate of FIG. 9, in accordance with the present disclosure;

FIG. 13 is a left side view of the exemplary orthopedic plate of FIG. 9, in accordance with the present disclosure;

FIG. 14 is a right side view of the exemplary orthopedic plate of FIG. 9, in accordance with the present disclosure;

FIG. 15 is a top view of the exemplary orthopedic plate of FIG. 9, in accordance with the present disclosure;

FIG. 16 is a bottom view of the exemplary orthopedic plate of FIG. 9, in accordance with the present disclosure;

FIG. 17 is a front, left side perspective view of an exemplary orthopedic plate, in accordance with the present disclosure;

FIG. 18 is a rear, right side perspective view of the exemplary orthopedic plate of FIG. 17, in accordance with the present disclosure;

FIG. 19 is a front view of the exemplary orthopedic plate of FIG. 17, in accordance with the present disclosure;

FIG. 20 is a rear view of the exemplary orthopedic plate of FIG. 17, in accordance with the present disclosure;

FIG. 21 is a left side view of the exemplary orthopedic plate of FIG. 17, in accordance with the present disclosure;

FIG. 22 is a right side view of the exemplary orthopedic plate of FIG. 17, in accordance with the present disclosure;

FIG. 23 is a top view of the exemplary orthopedic plate of FIG. 17, in accordance with the present disclosure;

FIG. 24 is a bottom view of the exemplary orthopedic plate of FIG. 17, in accordance with the present disclosure;

FIG. 25 is a front, left side perspective view of an exemplary orthopedic plate, in accordance with the present disclosure;

FIG. 26 is a rear, right side perspective view of the exemplary orthopedic plate of FIG. 25, in accordance with the present disclosure;

FIG. 27 is a front view of the exemplary orthopedic plate of FIG. 25, in accordance with the present disclosure;

FIG. 28 is a rear view of the exemplary orthopedic plate of FIG. 25, in accordance with the present disclosure;

FIG. 29 is a left side view of the exemplary orthopedic plate of FIG. 25, in accordance with the present disclosure;

FIG. 30 is a right side view of the exemplary orthopedic plate of FIG. 25, in accordance with the present disclosure;

FIG. 31 is a top view of the exemplary orthopedic plate of FIG. 25, in accordance with the present disclosure;

FIG. 32 is a bottom view of the exemplary orthopedic plate of FIG. 25, in accordance with the present disclosure;

FIG. 33 is a front, left side perspective view of an exemplary orthopedic plate, in accordance with the present disclosure;

FIG. 34 is a rear, right side perspective view of the exemplary orthopedic plate of FIG. 33, in accordance with the present disclosure;

FIG. 35 is a front view of the exemplary orthopedic plate of FIG. 33, in accordance with the present disclosure;

FIG. 36 is a rear view of the exemplary orthopedic plate of FIG. 33, in accordance with the present disclosure;

FIG. 37 is a left side view of the exemplary orthopedic plate of FIG. 33, in accordance with the present disclosure;

FIG. 38 is a right side view of the exemplary orthopedic plate of FIG. 33, in accordance with the present disclosure;

FIG. 39 is a top view of the exemplary orthopedic plate of FIG. 33, in accordance with the present disclosure;

FIG. 40 is a bottom view of the exemplary orthopedic plate of FIG. 33, in accordance with the present disclosure;

FIG. 41 is a front, left side perspective view of an exemplary orthopedic plate, in accordance with the present disclosure;

FIG. 42 is a rear, right side perspective view of the exemplary orthopedic plate of FIG. 41, in accordance with the present disclosure;

FIG. 43 is a front view of the exemplary orthopedic plate of FIG. 41, in accordance with the present disclosure;

FIG. 44 is a rear view of the exemplary orthopedic plate of FIG. 41, in accordance with the present disclosure;

FIG. 45 is a right side view of the exemplary orthopedic plate of FIG. 41, in accordance with the present disclosure;

FIG. 46 is a left side view of the exemplary orthopedic plate of FIG. 41, in accordance with the present disclosure;

FIG. 47 is a top view of the exemplary orthopedic plate of FIG. 41, in accordance with the present disclosure;

FIG. 48 is a bottom view of the exemplary orthopedic plate of FIG. 41, in accordance with the present disclosure;

FIG. 49 is a front, right side perspective view of an exemplary orthopedic instrument, in accordance with the present disclosure;

FIG. 50 is a rear, left side perspective view of the exemplary orthopedic instrument of FIG. 49, in accordance with the present disclosure;

FIG. 51 is a top view of the exemplary orthopedic instrument of FIG. 49, in accordance with the present disclosure;

FIG. 52 is a bottom view of the exemplary orthopedic instrument of FIG. 49, in accordance with the present disclosure;

FIG. 53 is a right side view of the exemplary orthopedic instrument of FIG. 49, in accordance with the present disclosure;

FIG. 54 is a left side view of the exemplary orthopedic instrument of FIG. 49, in accordance with the present disclosure;

FIG. 55 is a rear view of the exemplary orthopedic instrument of FIG. 49, in accordance with the present disclosure;

FIG. 56 is a front view of the exemplary orthopedic instrument of FIG. 49, in accordance with the present disclosure;

FIG. 57 is a rear, right side perspective view of an exemplary orthopedic instrument, in accordance with the present disclosure;

FIG. 58 is a rear, left side perspective view of the exemplary orthopedic instrument of FIG. 57, in accordance with the present disclosure;

FIG. 59 is a top view of the exemplary orthopedic instrument of FIG. 57, in accordance with the present disclosure;

FIG. 60 is a bottom view of the exemplary orthopedic instrument of FIG. 57, in accordance with the present disclosure;

FIG. 61 is a front view of the exemplary orthopedic instrument of FIG. 57, in accordance with the present disclosure;

FIG. 62 is a rear view of the exemplary orthopedic instrument of FIG. 57, in accordance with the present disclosure;

FIG. 63 is a front perspective view of the exemplary orthopedic instrument of FIG. 57 implemented in conjunction with the orthopedic implant of FIGS. 1-8, in accordance with the present disclosure;

FIG. 64 is a side perspective view of the exemplary orthopedic instrument of FIG. 57 implemented in conjunction with the orthopedic implant of FIGS. 1-8, in accordance with the present disclosure

FIG. 65 is a front, right side perspective view of an exemplary orthopedic instrument, in accordance with the present disclosure;

FIG. 66 is a rear, left side perspective view of the exemplary orthopedic instrument of FIG. 65, in accordance with the present disclosure;

FIG. 67 is a front view of the exemplary orthopedic instrument of FIG. 65, in accordance with the present disclosure;

FIG. 68 is a rear view of the exemplary orthopedic instrument of FIG. 65, in accordance with the present disclosure;

FIG. 69 is a left side view of the exemplary orthopedic instrument of FIG. 65, in accordance with the present disclosure;

FIG. 70 is a right side view of the exemplary orthopedic instrument of FIG. 65, in accordance with the present disclosure;

FIG. 71 is a top view of the exemplary orthopedic instrument of FIG. 65, in accordance with the present disclosure;

FIG. 72 is a bottom view of the exemplary orthopedic instrument of FIG. 65, in accordance with the present disclosure;

FIG. 73 is a front perspective view of the exemplary orthopedic instrument of FIG. 65 implemented in conjunction with the orthopedic implant of FIGS. 1-8, in accordance with the present disclosure;

FIG. 74 is a left side perspective view of the exemplary orthopedic instrument of FIG. 65 implemented in conjunction with the orthopedic implant of FIGS. 1-8, in accordance with the present disclosure;

FIG. 75 is a front, right side perspective view of an exemplary orthopedic instrument, in accordance with the present disclosure;

FIG. 76 is a rear, left side perspective view of the exemplary orthopedic instrument of FIG. 75, in accordance with the present disclosure;

FIG. 77 is a top view of the exemplary orthopedic instrument of FIG. 75, in accordance with the present disclosure;

FIG. 78 is a bottom view of the exemplary orthopedic instrument of FIG. 75, in accordance with the present disclosure;

FIG. 79 is a rear view of the exemplary orthopedic instrument of FIG. 75, in accordance with the present disclosure;

FIG. 80 is a front view of the exemplary orthopedic instrument of FIG. 75, in accordance with the present disclosure;

FIG. 81 is a right side view of the exemplary orthopedic instrument of FIG. 75, in accordance with the present disclosure; and

FIG. 82 is a left side view of the exemplary orthopedic instrument of FIG. 75, in accordance with the present disclosure.

### DETAILED DESCRIPTION OF THE INVENTION

In this detailed description and the following claims, the words proximal, distal, anterior or plantar, posterior or dorsal, medial, lateral, superior and inferior are defined by their standard usage for indicating a particular part or portion of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure. Further, specifically in regards to the foot, the term “dorsal” refers to the top of the foot and the term “plantar” refers to the bottom of the foot.

Similarly, positions or directions may be used herein with reference to anatomical structures or surfaces. For example, as the current implants, devices, instrumentation, and methods are described herein with reference to use with the bones of the foot, the bones of the foot, ankle and lower leg may be used to describe the surfaces, positions, directions or orientations of the implants, devices, instrumentation and methods. Further, the implants, devices, instrumentation, and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, instrumentation, and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, instrumentation, and methods, and the aspects, components, features and the like thereof, described herein with respect to the right foot may be mirrored so that they likewise function with the left foot. Further, the implants, devices, instrumentation, and methods, and the aspects, components, features and the like thereof, disclosed herein are described with respect to the foot for brevity purposes, but it should be understood that the implants, devices, instrumentation, and methods may be used with other bones of the body having similar structures.

The plates shown and described herein may be implemented, alone or with other implants, by a physician to treat fractures to the tibia of a patient. In some aspects, the plates shown and described herein may be implemented by a physician to treat fractures to the distal tibia of a patient, which may include fractures near the weight-bearing (or articulating) surface of the distal tibia adjacent to the ankle joint.

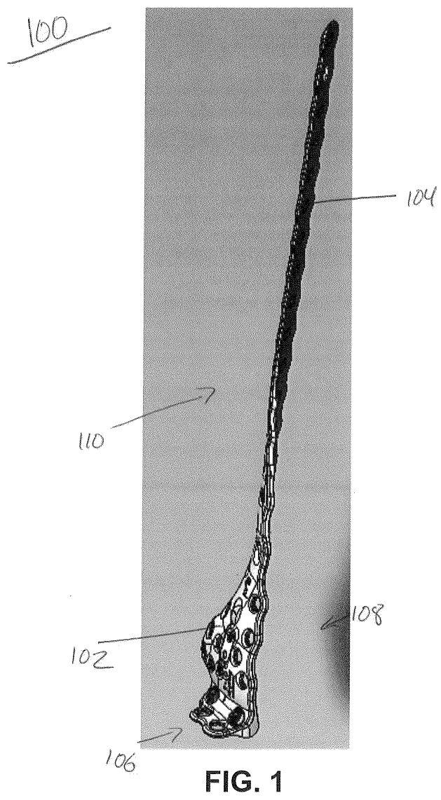

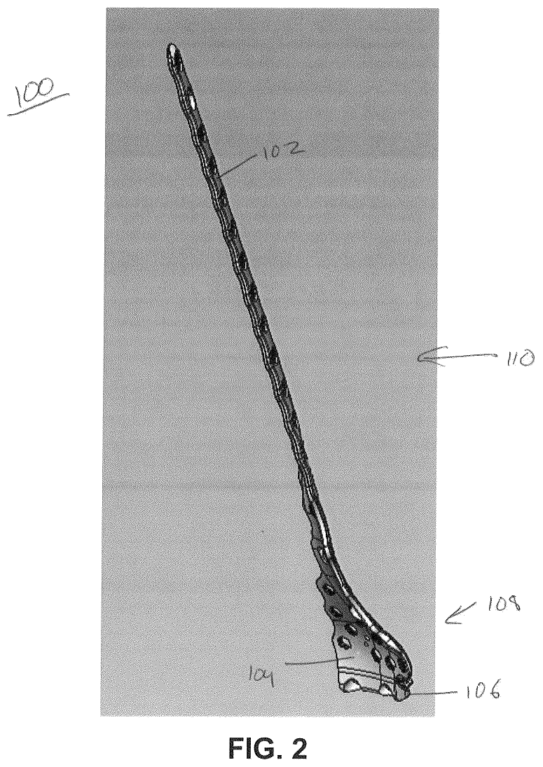

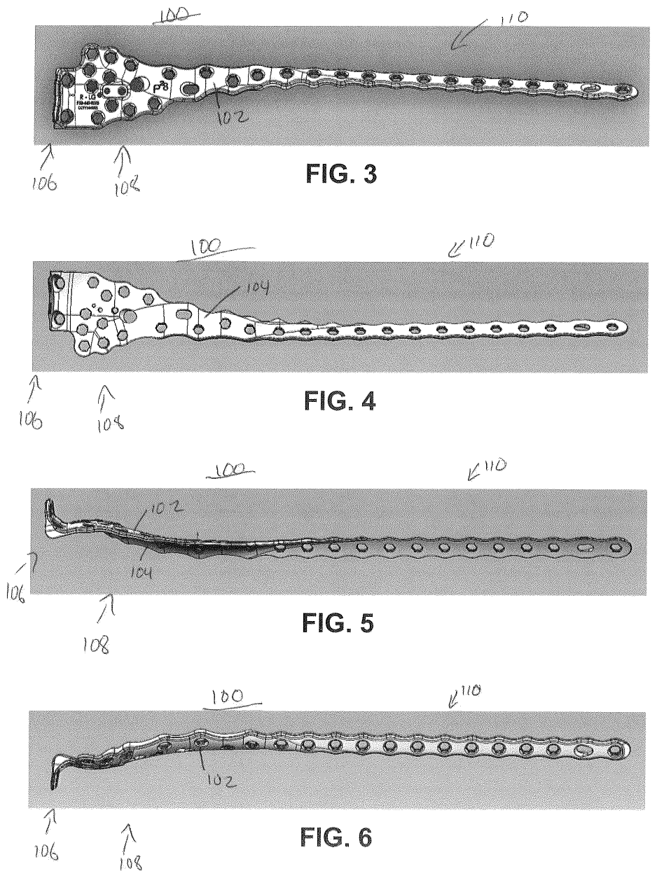

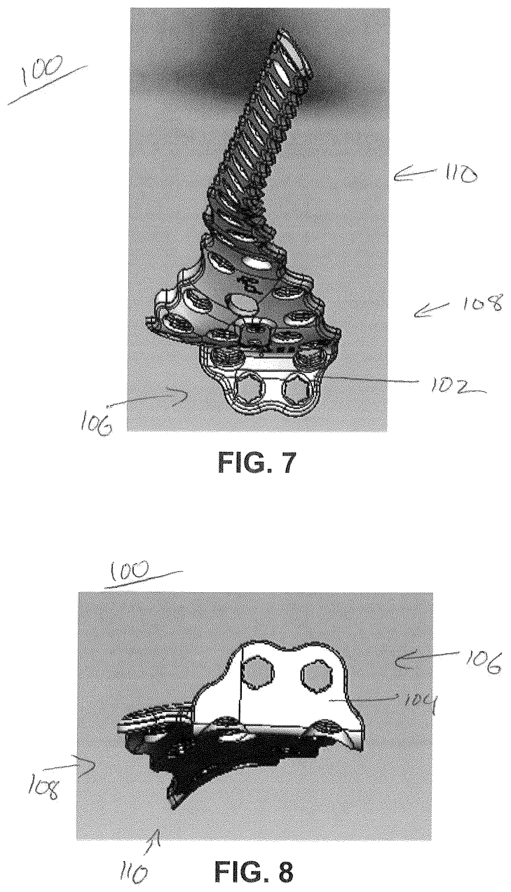

Referring now to FIGS. 1-8, a plate 100 is shown, according to an exemplary embodiment. The plate 100 shown herein may be configured to be implanted and secured to a right tibia of a patient, although similar plates with corresponding features for a left tibia of the patient are also contemplated. The plate 100 is shown to include an upper surface 102 positioned substantially opposite the plate 100 from a lower surface 104. Both the upper and lower surfaces 102, 104 may have various curvatures or increases/decreases in elevation along the length of the plate and between opposing lateral sides of the plate as the plate is contoured to a curvature of a distal tibia of the patient. The plate 100 is shown to include various through holes extending from the upper surface 102 through a thickness of the plate to the lower surface 104, thus establishing fluid communication therebetween. The through holes may be sized and configured to receive one or more fasteners therethrough to facilitate coupling of the plate 100 with the distal tibia of the patient. In some aspects, one or more of the through holes may include a threading disposed on an inner surface thereof configured to engage with the complimentary threading of the fastener when the fastener is received through the through hole.

The plate 100 is further shown to include a base 106, a body 108, and an extension 110 where the base, body, and extension 106, 108, 110 are all integral with one another and the upper and lower surfaces are continuous along the base, body, and extensions 106, 108, 110. As shown, the base 106 may extend from the body 108 at an angle, for example a substantially orthogonal angle (e.g., such that the upper surface 102 on the base 106 is positioned at least partially in a plane that is orthogonal to that of the body 108) with the curved transition portion between the base 106 and the body 108. The body 108 may have a greater lateral dimension than that of the base 106, with the lateral dimension of the body 108 increasing from the transition portion with the base 106 to a greatest point and then decreasing between the greatest point and a transition with the extension 110. The extension 110 may extend from a portion of the body 108 substantially opposite that of the base 106 and include a lateral dimension lesser than that of the base or body 106, 108. In some aspects, the extension 110 may include a twisted or partially helical geometry configured to abut the curvature of the distal tibia of the patient. Further, various portions of the base, body, and extension 106, 108, 110 may include one or more curvatures, radii, or other geometries configure to accommodate the curved anatomy of the distal tibia of the patient. In some aspects, the thickness of the plate 100 may vary along the length of the plate (e.g., the body 108 having a thickness greater than that of the body 106 or the extension 110).

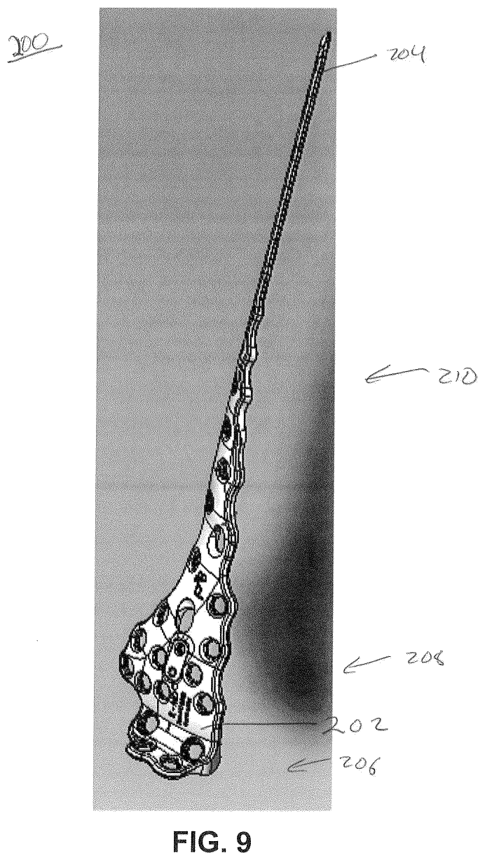

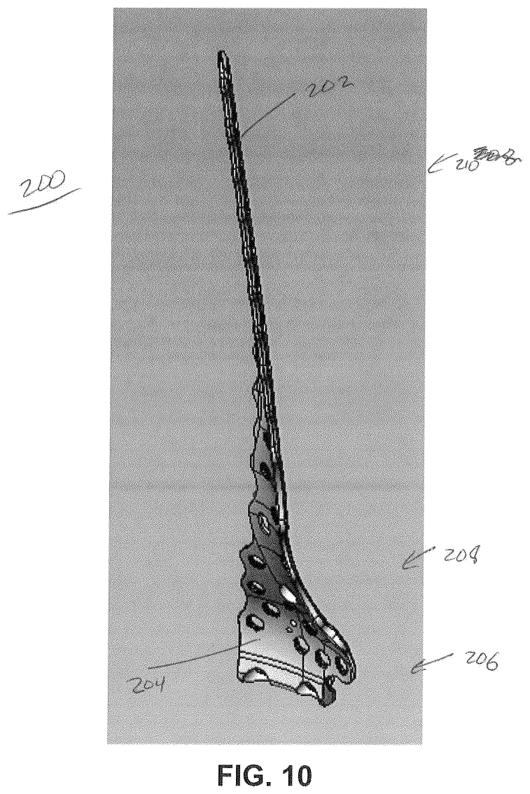

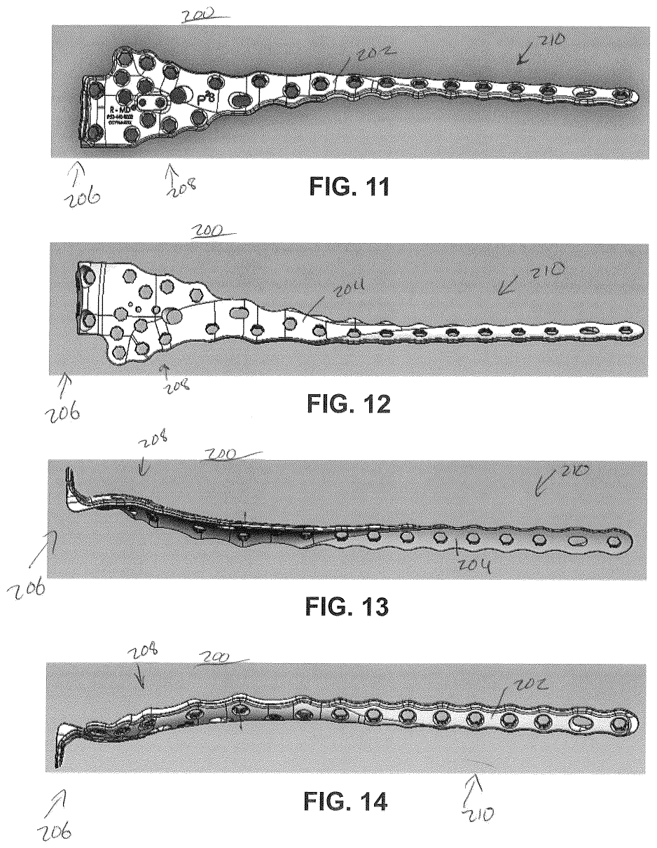

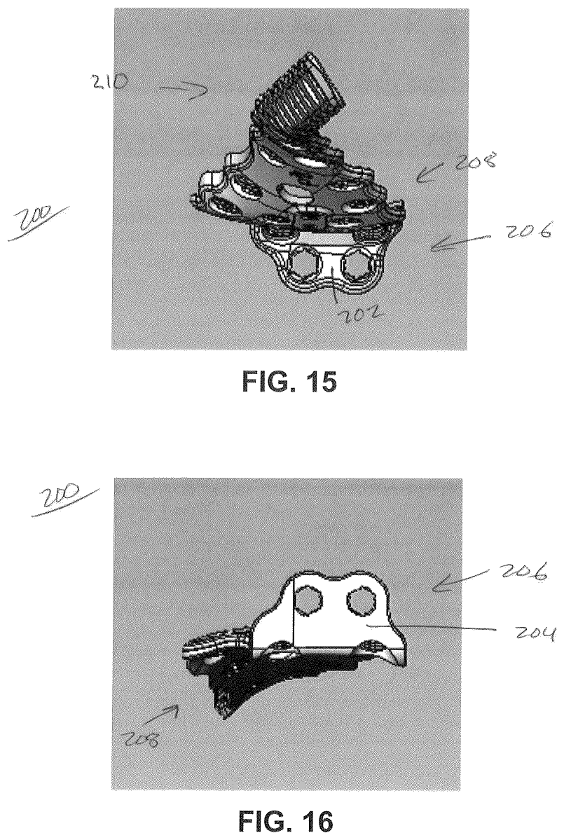

Referring now to FIGS. 9-16, a plate 200 is shown, according to an exemplary embodiment. The plate 200 shown herein may be configured to be implanted and secured to the right tibia of the patient, although similar plates with corresponding features for the left tibia of the patient are also contemplated. The plate 200 is shown to include an upper surface 202 positioned substantially opposite the plate 200 from a lower surface 204. Both the upper and lower surfaces 202, 204 may have various curvatures or increases/decreases in elevation along the length of the plate and between opposing lateral sides of the plate as the plate is contoured to a curvature of the distal tibia of the patient. The plate 200 is shown to include various through holes extending from the upper surface 202 through a thickness of the plate to the lower surface 204, thus establishing fluid communication therebetween. The through holes may be sized and configured to receive one or more fasteners therethrough to facilitate coupling of the plate 200 with the distal tibia of the patient. In some aspects, one or more of the through holes may include a threading disposed on an inner surface thereof configured to engage with the complimentary threading of the fastener when the fastener is received through the through hole.

The plate 200 is further shown to include a base 206, a body 208, and an extension 210 where the base, body, and extension 206, 208, 210 are all integral with one another and the upper and lower surfaces are continuous along the base, body, and extensions 206, 208, 210. As shown, the base 206 may extend from the body 208 at an angle, for example a substantially orthogonal angle (e.g., such that the upper surface 202 on the base 206 is positioned at least partially in a plane that is orthogonal to that of the body 208) with the curved transition portion between the base 206 and the body 208. The body 208 may have a greater lateral dimension than that of the base 206, with the lateral dimension of the body 208 increasing from the transition portion with the base 206 to a greatest point and then decreasing between the greatest point and a transition with the extension 210. The extension 210 may extend from a portion of the body 208 substantially opposite that of the base 206 and include a lateral dimension lesser than that of the base or body 206, 208. In some aspects, the extension 210 may include a twisted or partially helical geometry configured to abut the curvature of the distal tibia of the patient. Further, various portions of the base, body, and extension 206, 208, 210 may include one or more curvatures, radii, or other geometries configure to accommodate the curved anatomy of the distal tibia of the patient. In some aspects, the thickness of the plate 200 may vary along the length of the plate (e.g., the body 208 having a thickness greater than that of the body 206 or the extension 210). In some aspects, the extension 210 may be shorter than the extension 110 of the plate 100.

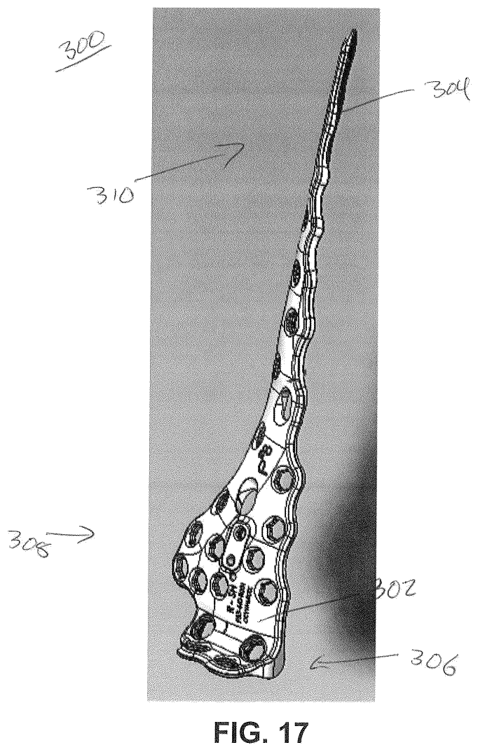

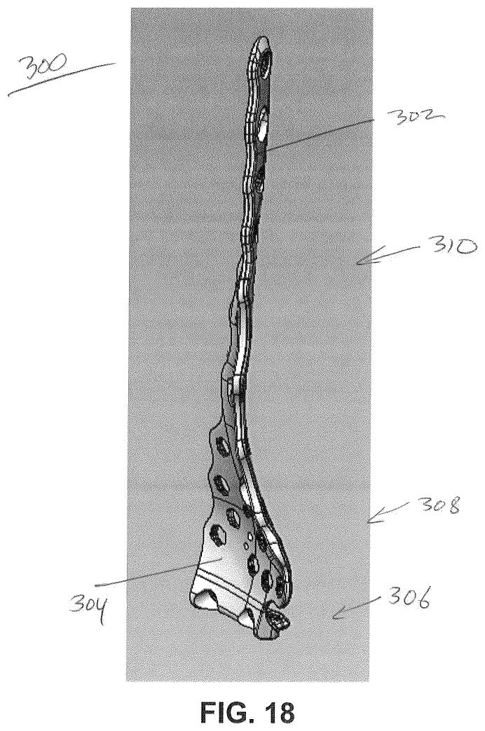

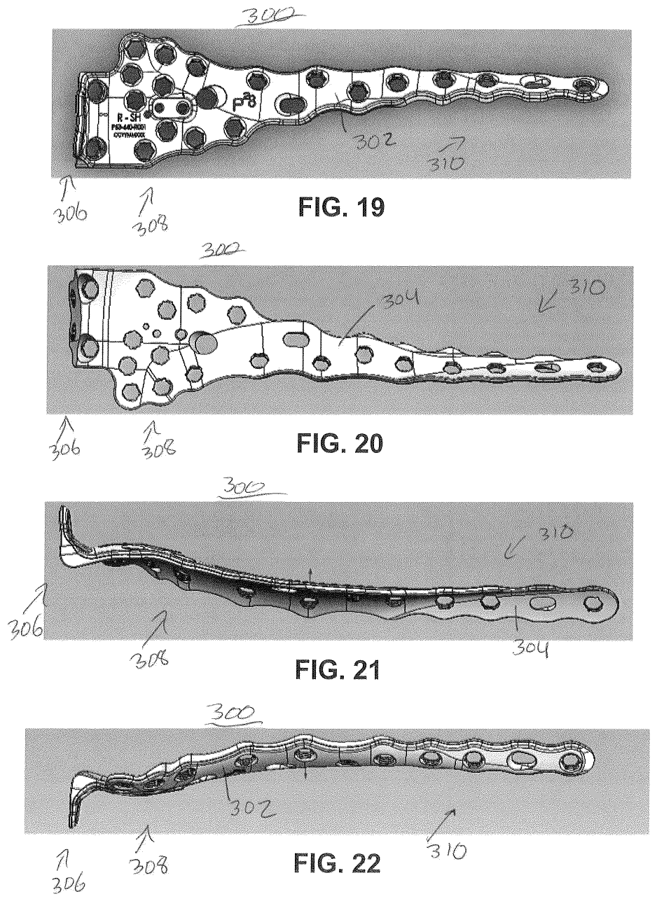

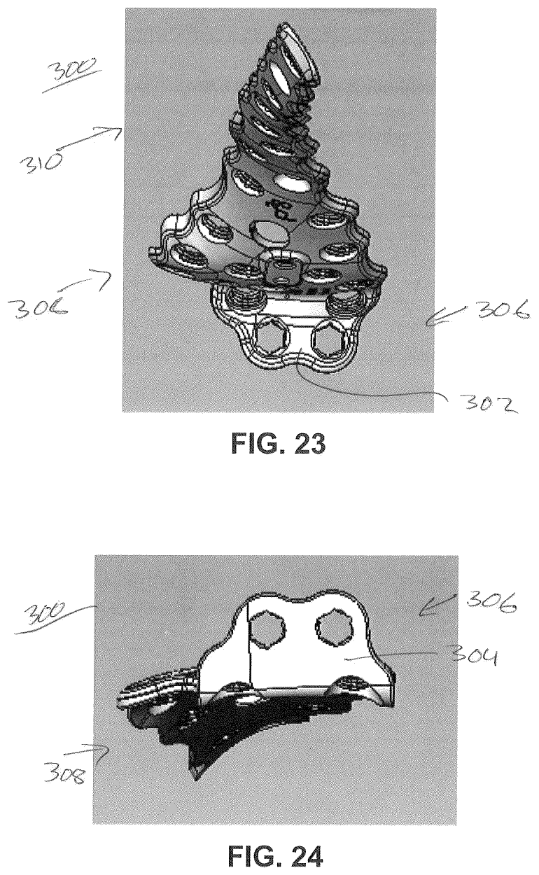

Referring now to FIGS. 17-24, a plate 300 is shown, according to an exemplary embodiment. The plate 300 shown herein may be configured to be implanted and secured to the right tibia of the patient, although similar plates with corresponding features for the left tibia of the patient are also contemplated. The plate 300 is shown to include an upper surface 302 positioned substantially opposite the plate 300 from a lower surface 304. Both the upper and lower surfaces 302, 304 may have various curvatures or increases/decreases in elevation along the length of the plate and between opposing lateral sides of the plate as the plate is contoured to a curvature of the distal tibia of the patient. The plate 300 is shown to include various through holes extending from the upper surface 302 through a thickness of the plate to the lower surface 304, thus establishing fluid communication therebetween. The through holes may be sized and configured to receive one or more fasteners therethrough to facilitate coupling of the plate 300 with the distal tibia of the patient. In some aspects, one or more of the through holes may include a threading disposed on an inner surface thereof configured to engage with the complimentary threading of the fastener when the fastener is received through the through hole.

The plate 300 is further shown to include a base 306, a body 308, and an extension 310 where the base, body, and extension 306, 308, 310 are all integral with one another and the upper and lower surfaces are continuous along the base, body, and extensions 306, 308, 310. As shown, the base 306 may extend from the body 308 at an angle, for example a substantially orthogonal angle (e.g., such that the upper surface 302 on the base 306 is positioned at least partially in a plane that is orthogonal to that of the body 308) with the curved transition portion between the base 306 and the body 308. The body 308 may have a greater lateral dimension than that of the base 306, with the lateral dimension of the body 308 increasing from the transition portion with the base 306 to a greatest point and then decreasing between the greatest point and a transition with the extension 310. The extension 310 may extend from a portion of the body 308 substantially opposite that of the base 306 and include a lateral dimension lesser than that of the base or body 306, 308. In some aspects, the extension 310 may include a twisted or partially helical geometry configured to abut the curvature of the distal tibia of the patient. Further, various portions of the base, body, and extension 306, 308, 310 may include one or more curvatures, radii, or other geometries configure to accommodate the curved anatomy of the distal tibia of the patient. In some aspects, the thickness of the plate 300 may vary along the length of the plate (e.g., the body 308 having a thickness greater than that of the body 306 or the extension 310). In some aspects, the extension 310 may be shorter than the extensions 110, 210 of the plates 100, 200.

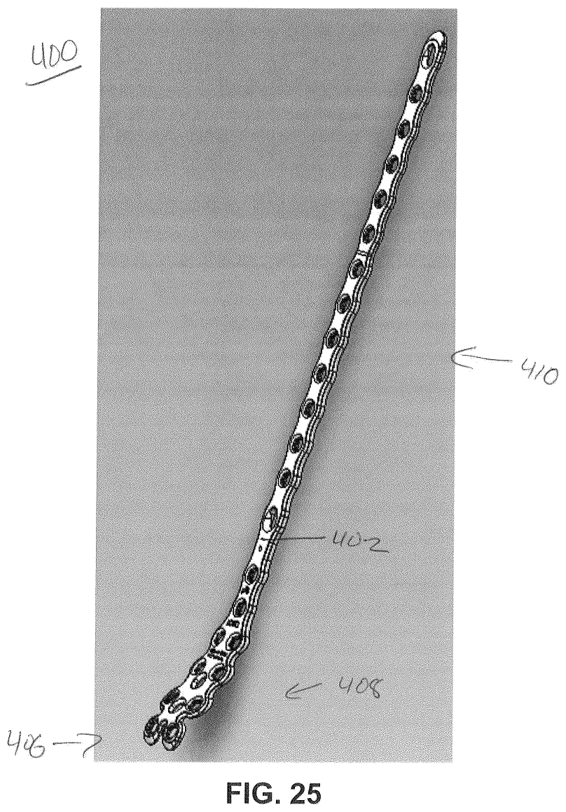

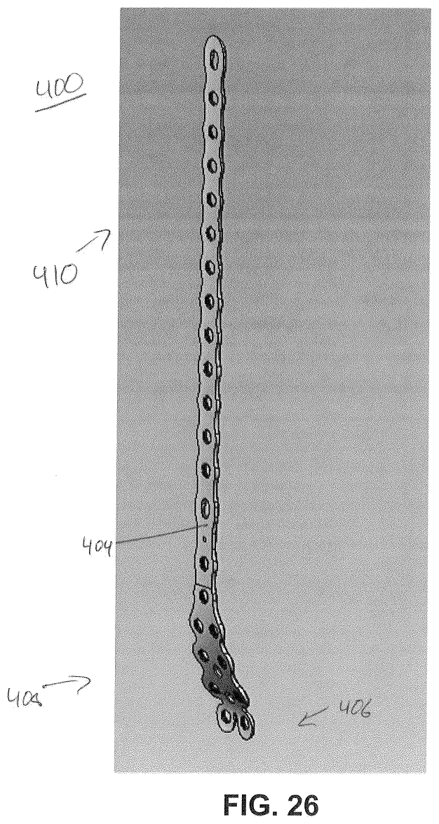

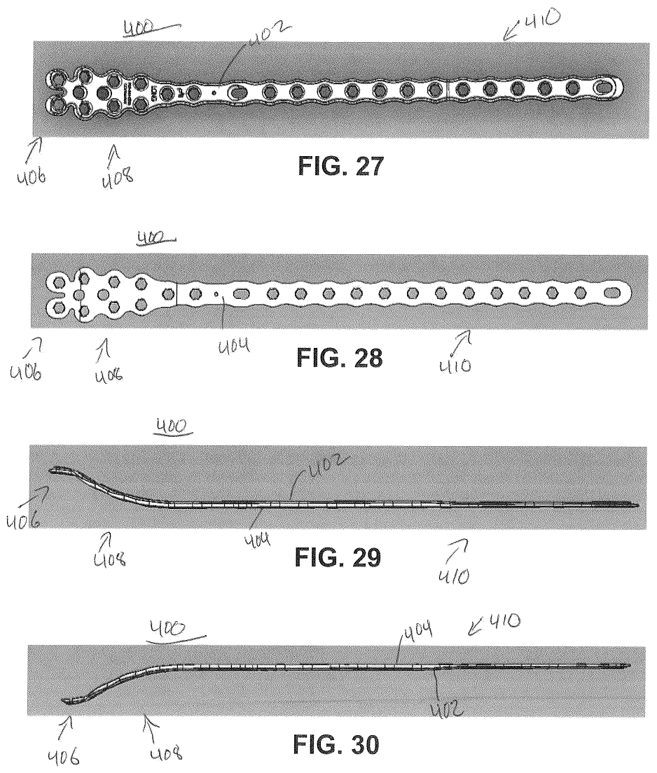

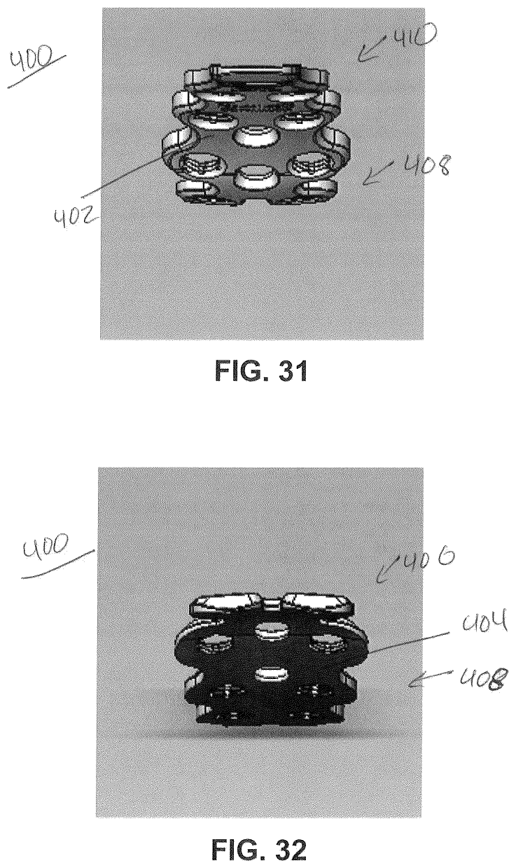

Referring now to FIGS. 25-32, a plate 400 is shown, according to an exemplary embodiment. The plate 400 shown herein may be configured to be implanted and secured to the right or left tibia of the patient. The plate 400 is shown to include an upper surface 402 positioned substantially opposite the plate 400 from a lower surface 404. Both the upper and lower surfaces 402, 404 may have various curvatures or increases/decreases in elevation along the length of the plate and between opposing lateral sides of the plate as the plate is contoured to a curvature of the distal tibia of the patient. The plate 400 is shown to include various through holes extending from the upper surface 402 through a thickness of the plate to the lower surface 404, thus establishing fluid communication therebetween. The through holes may be sized and configured to receive one or more fasteners therethrough to facilitate coupling of the plate 400 with the distal tibia of the patient. In some aspects, one or more of the through holes may include a threading disposed on an inner surface thereof configured to engage with the complimentary threading of the fastener when the fastener is received through the through hole.

The plate 400 is further shown to include a base 406, a body 408, and an extension 410 where the base, body, and extension 406, 408, 410 are all integral with one another and the upper and lower surfaces are continuous along the base, body, and extensions 406, 408, 410. As shown, the base 406 may extend from the body 408 at an angle or along a radius, for example a radius of curvature or a substantially orthogonal angle or (e.g., such that the upper surface 402 on the base 406 is positioned at least partially in a plane that is orthogonal to that of the body 408) with the curved transition portion between the base 406 and the body 408. The body 408 may have a greater lateral dimension than that of the base 406, with the lateral dimension of the body 408 increasing from the transition portion with the base 406 to a greatest point and then decreasing between the greatest point and a transition with the extension 410. The extension 410 may extend from a portion of the body 408 substantially opposite that of the base 406 at an oblique angle (e.g., such that the top surface 402 on the extension 408 is positioned at least partially in a plane that forms an oblique angle with the top surface 402 of the body 408) and include a lateral dimension lesser than that of the base or body 406, 408. In some aspects, the extension 410 may include a twisted or partially helical geometry configured to abut the curvature of the distal tibia of the patient. Further, various portions of the base, body, and extension 406, 408, 410 may include one or more curvatures, radii, or other geometries configure to accommodate the curved anatomy of the distal tibia of the patient. In some aspects, the thickness of the plate 400 may vary along the length of the plate (e.g., the body 408 having a thickness greater than that of the body 406 or the extension 410).

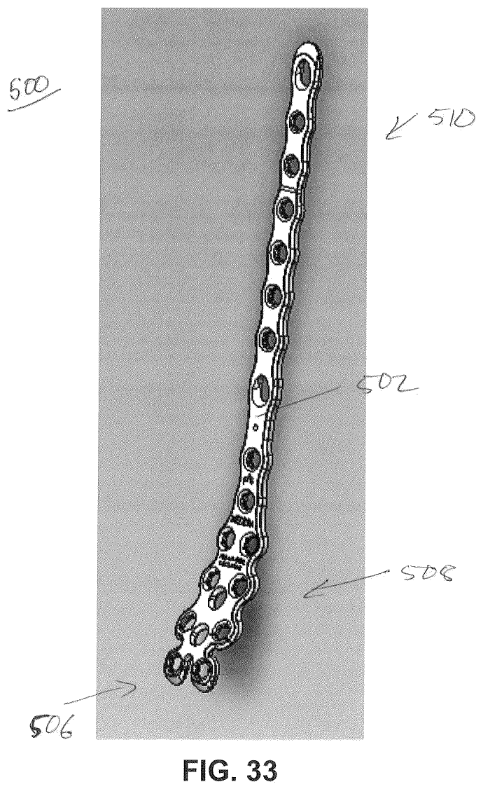

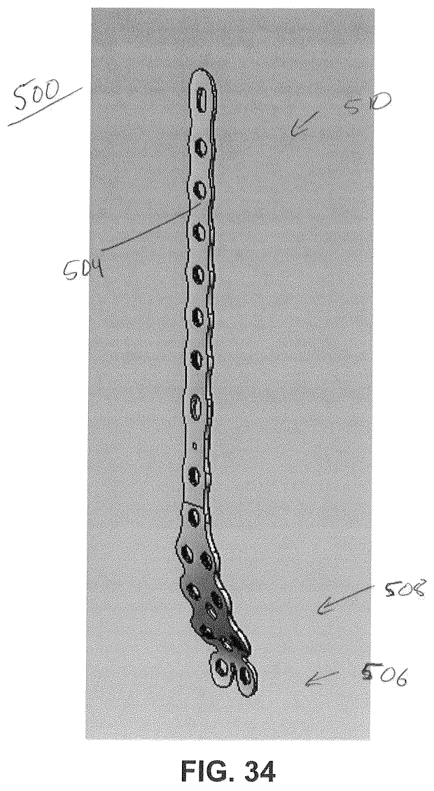

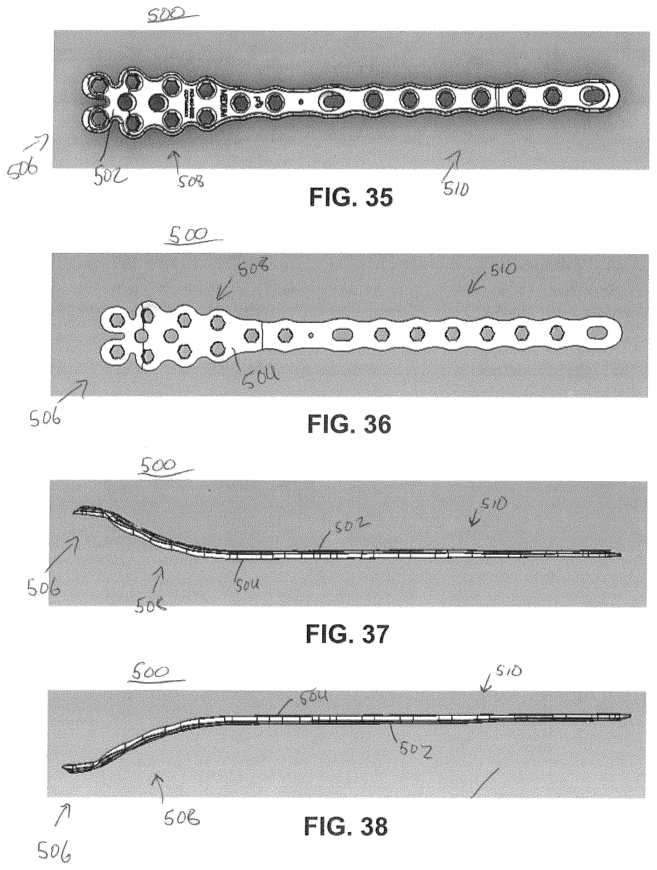

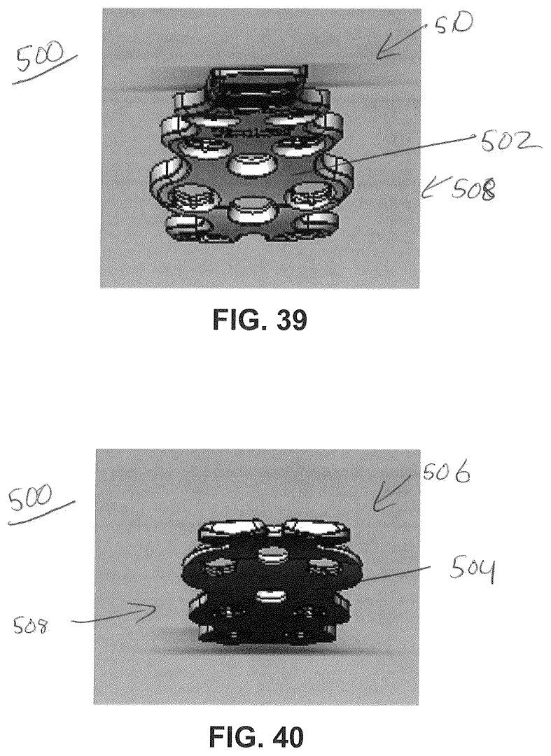

Referring now to FIGS. 33-40, a plate 500 is shown, according to an exemplary embodiment. The plate 500 shown herein may be configured to be implanted and secured to the right or left tibia of the patient. The plate 500 is shown to include an upper surface 502 positioned substantially opposite the plate 500 from a lower surface 504. Both the upper and lower surfaces 502, 504 may have various curvatures or increases/decreases in elevation along the length of the plate and between opposing lateral sides of the plate as the plate is contoured to a curvature of a distal tibia of the patient. The plate 500 is shown to include various through holes extending from the upper surface 502 through a thickness of the plate to the lower surface 504, thus establishing fluid communication therebetween. The through holes may be sized and configured to receive one or more fasteners therethrough to facilitate coupling of the plate 500 with the distal tibia of the patient. In some aspects, one or more of the through holes may include a threading disposed on an inner surface thereof configured to engage with the complimentary threading of the fastener when the fastener is received through the through hole.

The plate 500 is further shown to include a base 506, a body 508, and an extension 510 where the base, body, and extension 506, 508, 510 are all integral with one another and the upper and lower surfaces are continuous along the base, body, and extensions 506, 508, 510. As shown, the base 506 may extend from the body 508 at an angle or along a radius, for example a radius of curvature or a substantially orthogonal angle or (e.g., such that the upper surface 502 on the base 506 is positioned at least partially in a plane that is orthogonal to that of the body 508) with the curved transition portion between the base 506 and the body 508. The body 508 may have a greater lateral dimension than that of the base 506, with the lateral dimension of the body 508 increasing from the transition portion with the base 506 to a greatest point and then decreasing between the greatest point and a transition with the extension 510. The extension 510 may extend from a portion of the body 508 substantially opposite that of the base 506 at an oblique angle (e.g., such that the top surface 502 on the extension 508 is positioned at least partially in a plane that forms an oblique angle with the top surface 502 of the body 508) and include a lateral dimension lesser than that of the base or body 506, 508. In some aspects, the extension 510 may include a twisted or partially helical geometry configured to abut the curvature of the distal tibia of the patient. Further, various portions of the base, body, and extension 506, 508, 510 may include one or more curvatures, radii, or other geometries configure to accommodate the curved anatomy of the distal tibia of the patient. In some aspects, the thickness of the plate 500 may vary along the length of the plate (e.g., the body 508 having a thickness greater than that of the body 506 or the extension 510).

Referring now to FIGS. 25-32, a plate 600 is shown, according to an exemplary embodiment. The plate 600 shown herein may be configured to be implanted and secured to the right or left tibia of the patient. The plate 600 is shown to include an upper surface 602 positioned substantially opposite the plate 600 from a lower surface 604. Both the upper and lower surfaces 602, 604 may have various curvatures or increases/decreases in elevation along the length of the plate and between opposing lateral sides of the plate as the plate is contoured to a curvature of a distal tibia of the patient. The plate 600 is shown to include various through holes extending from the upper surface 602 through a thickness of the plate to the lower surface 604, thus establishing fluid communication therebetween. The through holes may be sized and configured to receive one or more fasteners therethrough to facilitate coupling of the plate 600 with the distal tibia of the patient. In some aspects, one or more of the through holes may include a threading disposed on an inner surface thereof configured to engage with the complimentary threading of the fastener when the fastener is received through the through hole.

The plate 600 is further shown to include a base 606, a body 608, and an extension 610 where the base, body, and extension 606, 608, 610 are all integral with one another and the upper and lower surfaces are continuous along the base, body, and extensions 606, 608, 610. As shown, the base 606 may extend from the body 608 at an angle or along a radius, for example a radius of curvature or a substantially orthogonal angle or (e.g., such that the upper surface 602 on the base 606 is positioned at least partially in a plane that is orthogonal to that of the body 608) with the curved transition portion between the base 606 and the body 608. The body 608 may have a greater lateral dimension than that of the base 606, with the lateral dimension of the body 608 increasing from the transition portion with the base 606 to a greatest point and then decreasing between the greatest point and a transition with the extension 610. The extension 610 may extend from a portion of the body 608 substantially opposite that of the base 606 at an oblique angle (e.g., such that the top surface 602 on the extension 608 is positioned at least partially in a plane that forms an oblique angle with the top surface 602 of the body 608) and include a lateral dimension lesser than that of the base or body 606, 608. In some aspects, the extension 610 may include a twisted or partially helical geometry configured to abut the curvature of the distal tibia of the patient. Further, various portions of the base, body, and extension 606, 608, 610 may include one or more curvatures, radii, or other geometries configure to accommodate the curved anatomy of the distal tibia of the patient. In some aspects, the thickness of the plate 600 may vary along the length of the plate (e.g., the body 608 having a thickness greater than that of the body 606 or the extension 610).

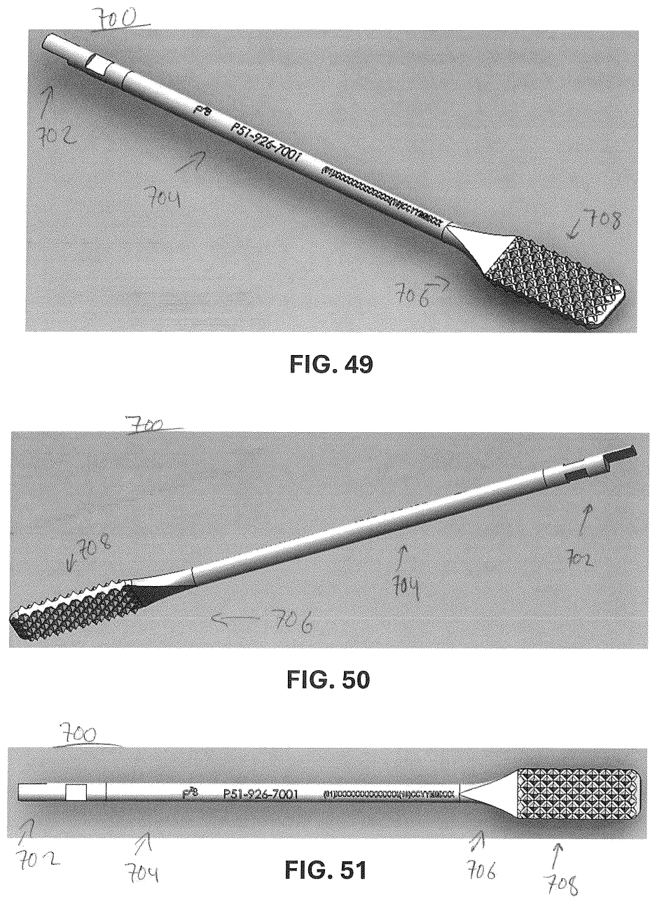

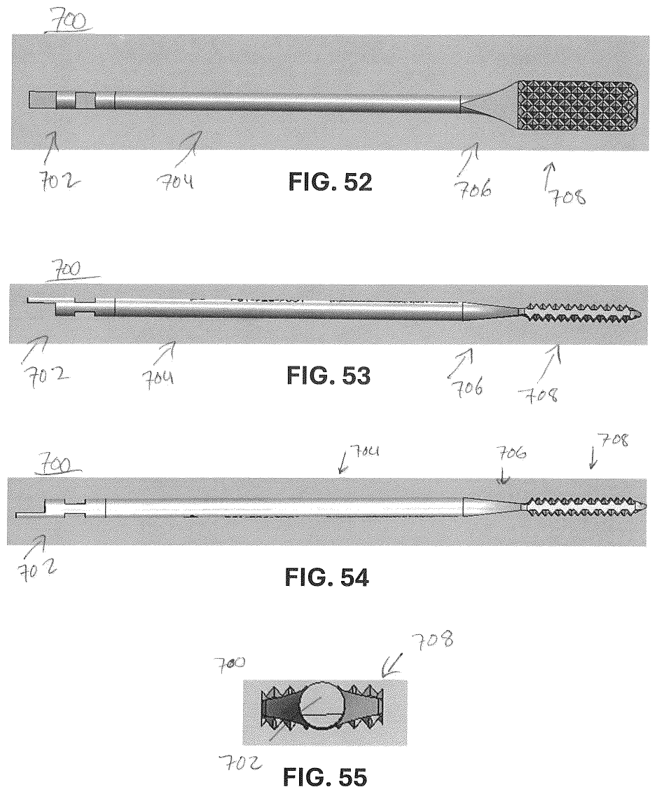

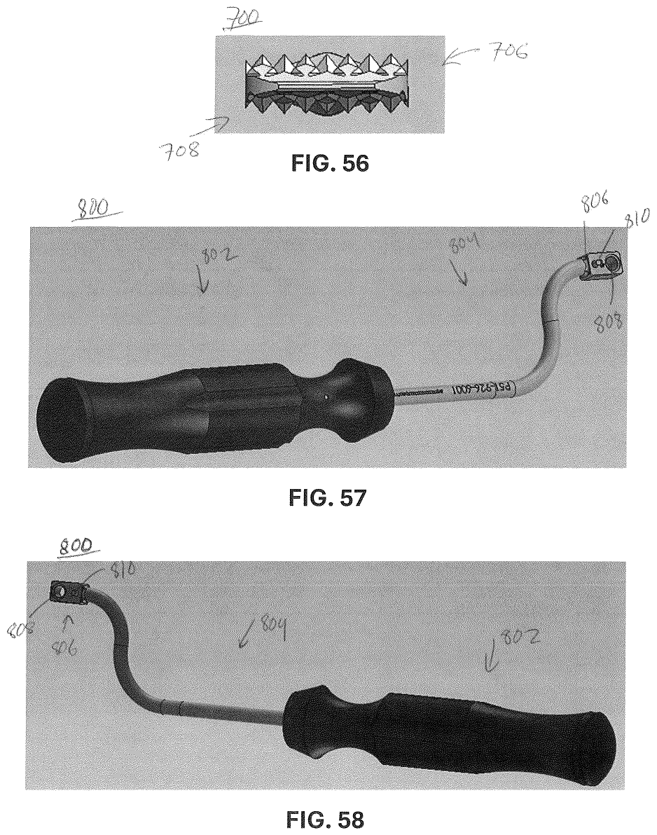

Referring now to FIGS. 49-56, an instrument 700 is shown, according to an exemplary embodiment. The instrument 700 is shown to include a first end 702 which includes a coupling interface. In some aspects, the coupling interface of the first end 702 may be configured to facilitate coupling with various powered instruments, for example to provide powered sagittal motion (e.g., like a sagittal saw) and/or reciprocating motion (e.g.,, like a reciprocating saw). Further, the coupling interface of the first end 702 may be a universal coupling interface configured to facilitate coupling with various other orthopedic instruments.

The instrument 700 is shown to include a shaft 704 extending from the first end 702, with the first end 702 opposite the shaft 704 from a second end 706. As shown, the shaft 704 is substantially cylindrical but in some aspects may have alternate geometries. The second end 706 is shown to include a width that is greater than that of the shaft 704 (e.g., greater than the diameter of the shaft 704). The second end is further shown to include at least one textured surface 708, which is shown in the exemplary embodiment of FIGS. 49-56 as plurality of projections extending perpendicular to the surface of the second end 706. In some aspects, the projections may have the same geometry (e.g., height, width, shape, etc.), however in some aspects the projections may vary in geometry. In some aspects, such as that shown in FIGS. 49-56, the textured surface 708 may be disposed on top and bottom surfaces of the second end 706, however in some embodiments the textured surface 708 may be positioned on only a top or bottom surface of the second end 706.

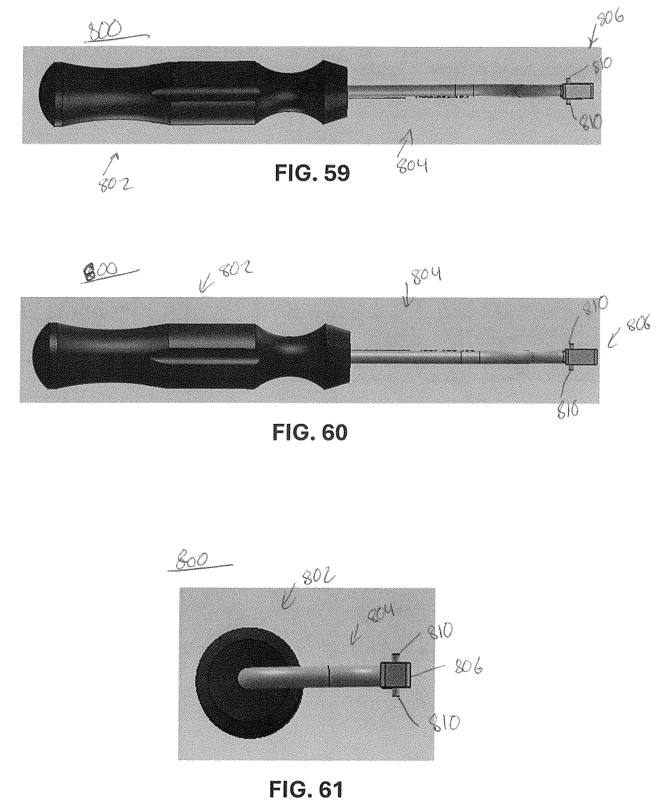

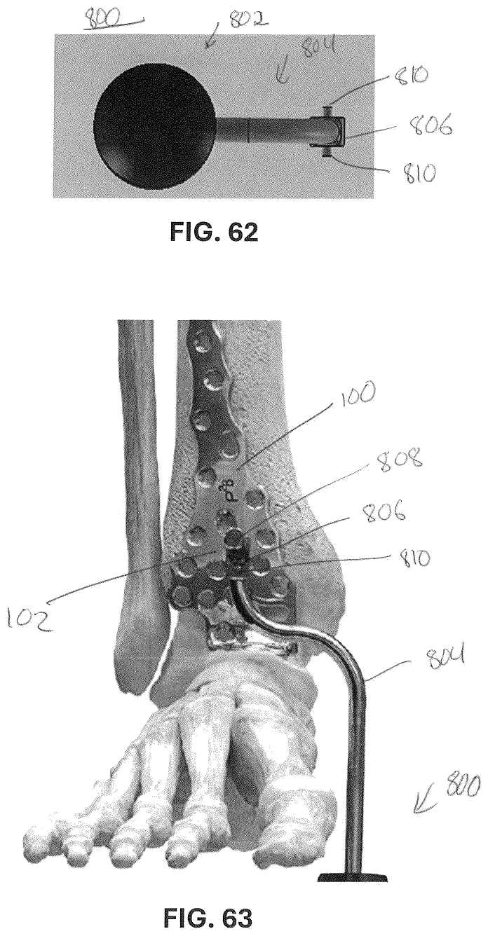

Referring now to FIGS. 57-64, an instrument 800 is shown, according to an exemplary embodiment. The instrument 800 is shown to include a first end 802 which includes a handle portion. In some aspects, the handle portion of the first end 802 may include one or more tapers or other ergonomic features to facilitate gripping, twisting, or other manipulation by a user.

The instrument 800 is shown to include a shaft 804 extending from the first end 802, with the first end 802 opposite the shaft 804 from a second end 806. As shown, the shaft 804 is substantially cylindrical but in some aspects may have alternate geometries. As shown in FIGS. 57-64, the shaft 804 includes a pair of bends (approximately 90 degrees) along the length thereof. However, in some aspects the shaft may be substantially linear or include a greater or fewer number of bends than those shown in the exemplary embodiment shown herein.

The second end 806 of the instrument 800 is shown to include a substantially rectangular prism-shaped geometry, although in alternate embodiments the second end 806 may include other, alternate geometries that differ from that of the shaft 804. The second end 806 is shown to include an opening 808 extending from a first surface through the second end 706 to a second surface, where the opening 808 is a substantially cylindrically-shaped opening. The second end 806 is further shown to include at least one projection 810 (shown in the exemplary embodiment of FIGS. 57-64 as a pair of projections) extending from at least one of the first and second surfaces of the second end 806.

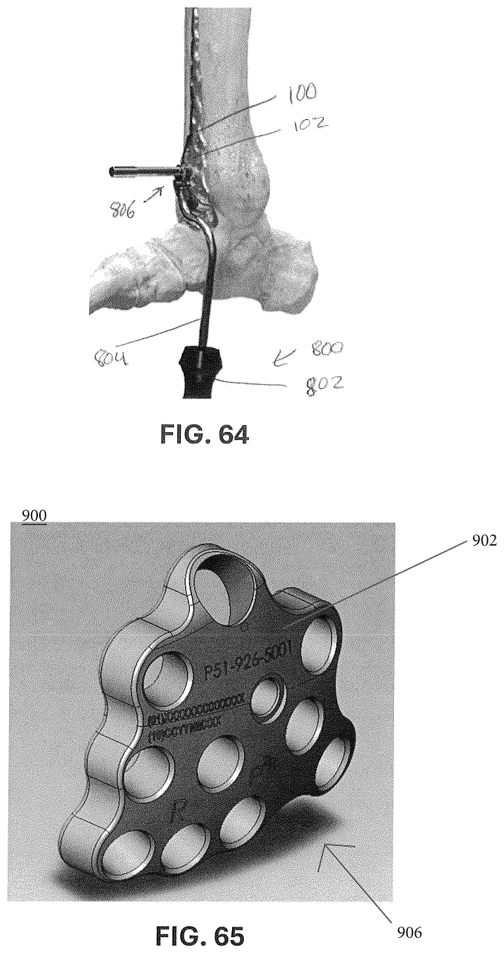

Referring now to FIGS. 63-64, the instrument 800 is shown to be implemented in conjunction with the plate 100 as shown and described previously herein, although the instrument 800 may be configured to be implemented in conjunction with various implants including but not limited to other plates shown and described herein. In some aspects, one or both of the opening 808 and the at least one projection of the second end 806 of the instrument 800 may be configured to facilitate coupling with the first surface 102 of the plate 100. For example, the plate 100 may include one or more openings configured to be coaxial with the opening 808 (e.g., so as to receive a coupling fastener therethrough to facilitate coupling) and/or may include one or more openings configured to receive at least a portion of the at least one projection 810 (to similarly facilitate coupling). A physician may grasp the handle of the first end 802 of the instrument 800 to facilitate manipulating and placing the plate 100 (e.g., on/adjacent to a distal portion of a tibia of a patient) when the plate 100 is releasably coupled with the second end 806 of the instrument 800.

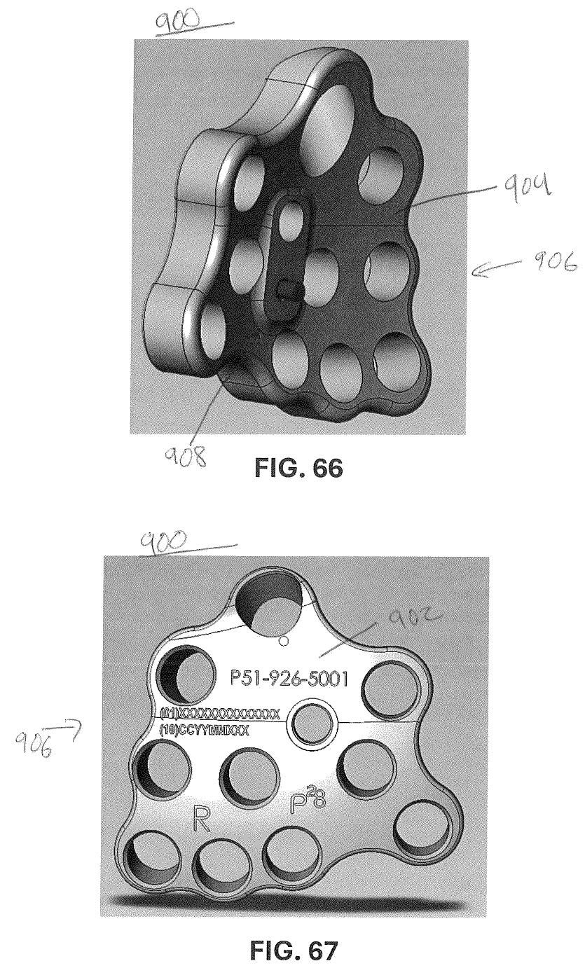

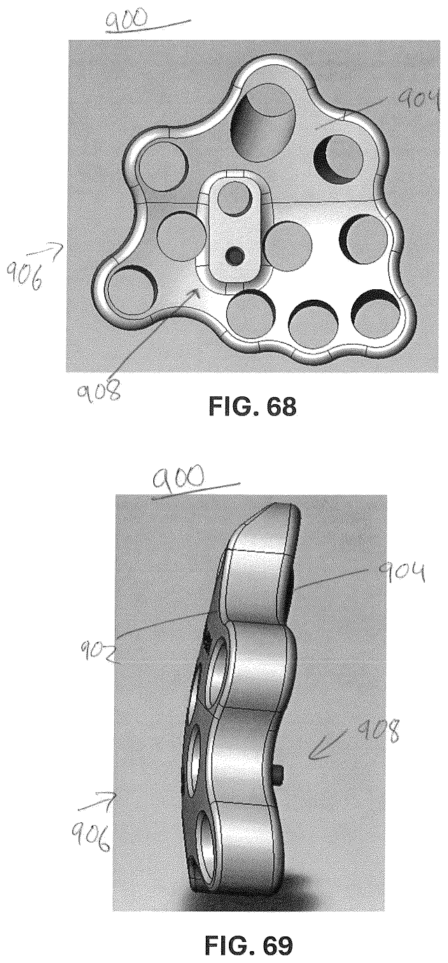

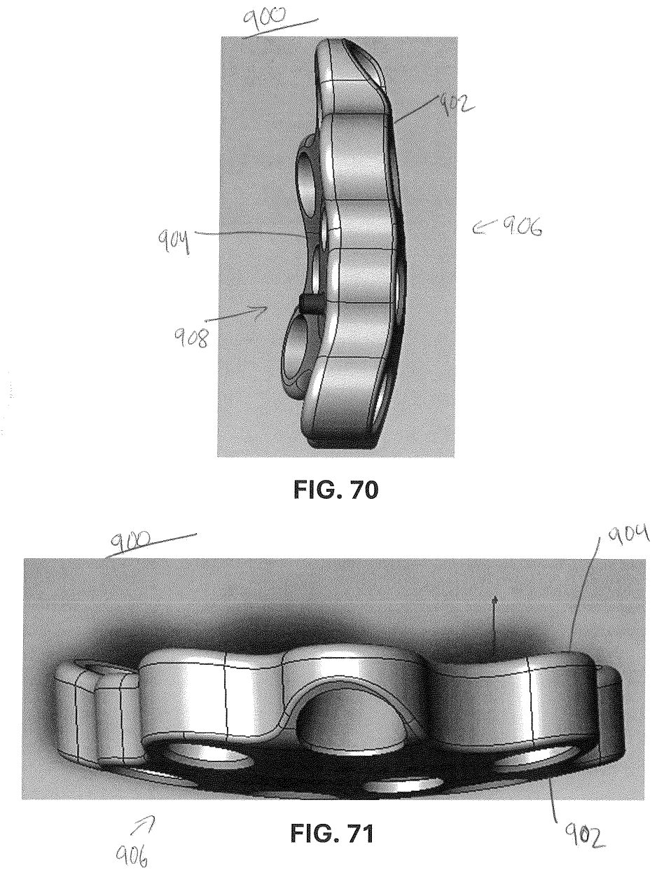

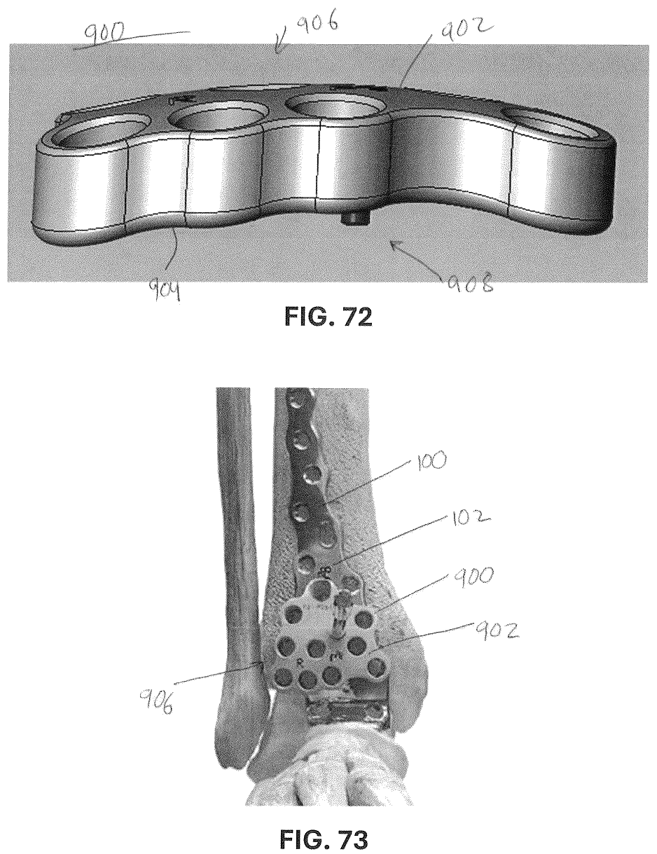

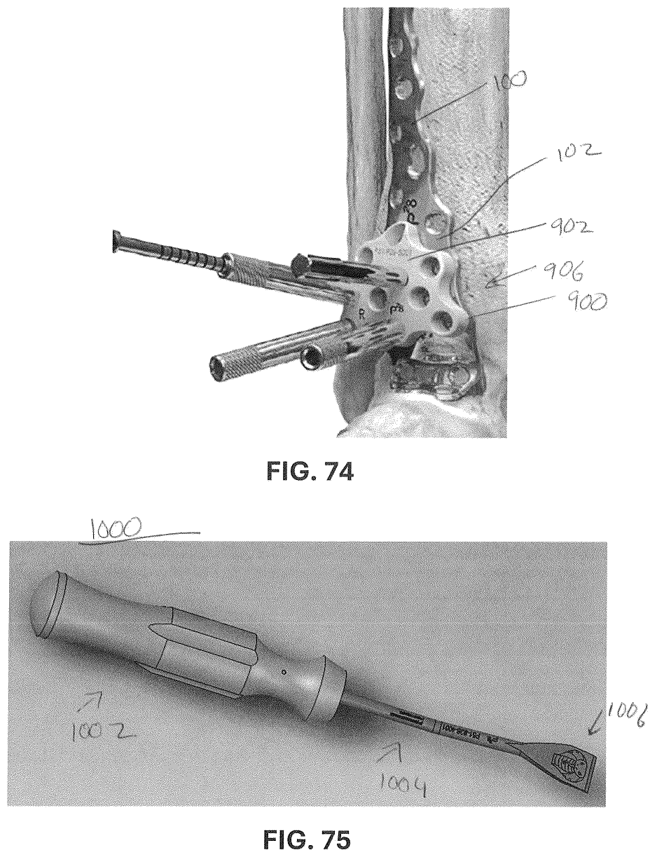

Referring now to FIGS. 65-74, an instrument 900 is shown, according to an exemplary embodiment. The instrument 900 may be implemented in conjunction with the plate 100 (e.g., as shown in FIGS. 73-74, with any of the other plates shown and described previously herein, or with other orthopedic implants) as well as other orthopedic instruments. The instrument 900 is shown to include a front surface 902 opposite the instrument 900 from a rear surface 904. The instrument 900 is further shown to include substantially convex geometry on the front surface 902 and a substantially concave geometry on the rear surface 904.

The instrument 900 is further shown to include a plurality of openings 906 extending from the front surface 902 through the instrument 900 to the rear surface 904, establishing fluid communication therebetween. As shown, each opening of the plurality of openings 906 has a substantially cylindrical geometry. In some aspects, the openings of the plurality of openings 906 may vary in geometry and/or diameter. Further, at least some openings of the plurality of openings 906 may have axes that form oblique angles with one another at some point beyond the instrument 900. The instrument 900 is further shown to include a coupling feature 908 extending from the rear surface 904. As shown, the coupling feature 908 includes a protrusion having a substantially cylindrical body which may facilitate releasable coupling of the instrument 900 with other instruments and/or implants.

As shown in the exemplary embodiment of FIGS. 73-74, the instrument 900 may be releasably couplable with an implant such as the plate 100. For example, the plate 100 may include an opening disposed on the upper surface 102 thereof that is configured to receive at least a portion of the coupling feature 908 therein. In some aspects, the instrument 900 may be configured such that when the coupling feature 908 is engaged with the plate 100, at least one of the plurality of openings 906 is positioned coaxially to at least one of the through holes of the plate 100. Accordingly, a physician may engage one or more other surgical instruments (e.g., k-wires, drill guides, targeting guides, etc.) and/or implants (e.g., fasteners, screws, etc.) with the instrument 900 and the plate 100. In some aspects, such other surgical instruments and/or implants may also be engaged with bony anatomy of the patient, for example the distal tibia or talus. With the instrument 900 releasably coupled with the plate 100 (at which point the instrument 900 and/or plate 100 may also be coupled with bony anatomy of the patient), a physician may place one or more instruments at least partially through at least one of the plurality of openings 906 and at least one of the through holes of the plate 100. In some aspects, a physician may implement the instrument 900 to guide the creation of pilot holes or the placement of temporary fixation of the plate 100 with bony anatomy of a patient before decoupling the instrument 900 from the plate 100 and placing permanent fixation (e.g., screws/fasteners) of the plate 100 to the bony anatomy.

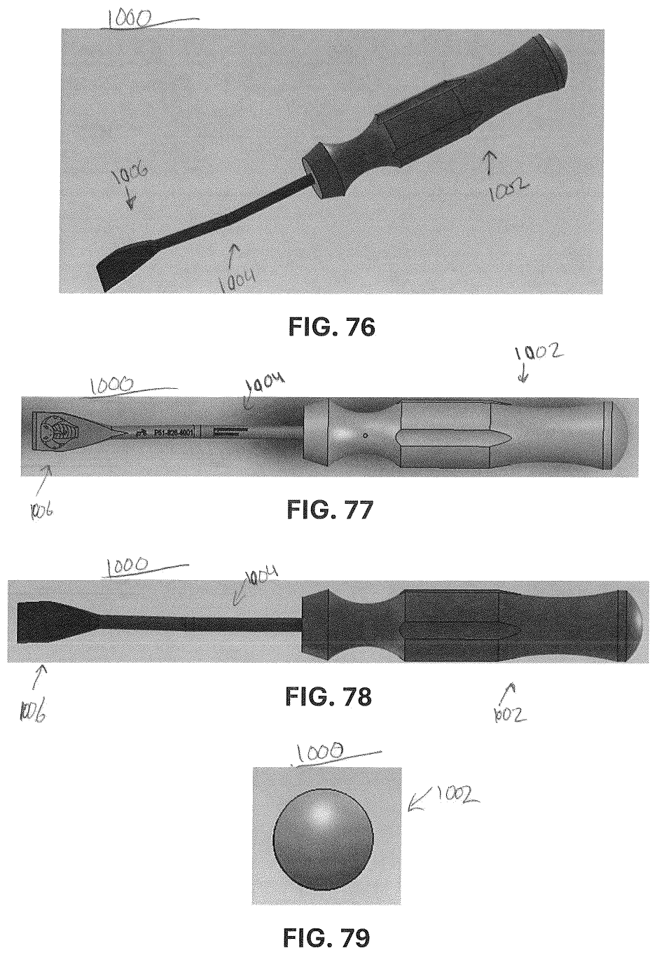

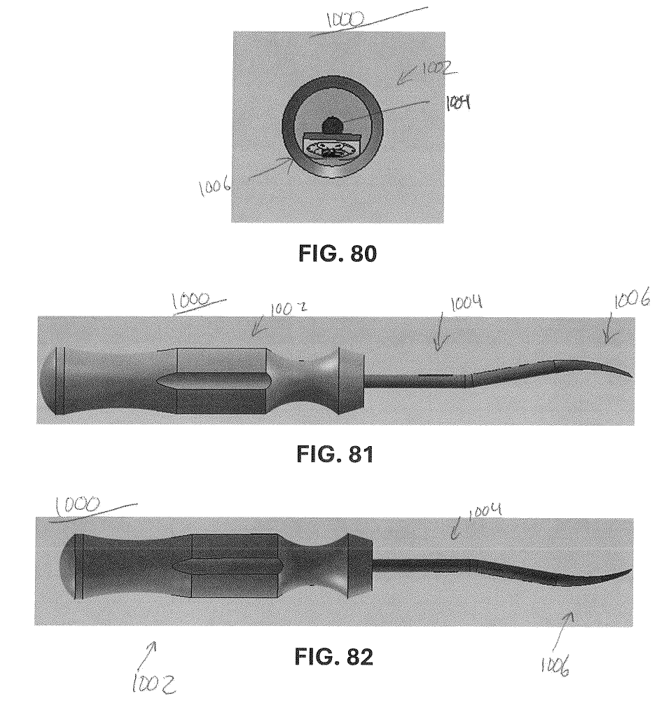

Referring now to FIGS. 75-82, an instrument 1000 is shown, according to an exemplary embodiment. The instrument 1000 is shown to include a first end 1002 which includes a handle portion. In some aspects, the handle portion of the first end 1002 may include one or more tapers or other ergonomic features to facilitate gripping, twisting, or other manipulation by a user.

The instrument 1000 is shown to include a shaft 1004 extending from the first end 1002, with the first end 1002 opposite the shaft 1004 from a second end 1006. As shown, the shaft 1004 is substantially cylindrical and has a lesser lateral dimension than that of the first end 1002, but in some aspects may have alternate geometries. The second end 1006 of the instrument 1000 is shown to have a lateral dimension that is greater in at least one plane than that of the shaft 1004. In some aspects, the second end 1006 may include a curvature along at least a portion of a length thereof. The second end 1006, as shown in FIGS. 75-82, also may include a taper from a point adjacent the shaft 1004 to a terminal end thereof, thus forming a cutting or scraping edge at the terminal end of the second end 1006.

The terminology used herein for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way but may also be configured in ways that are not listed.

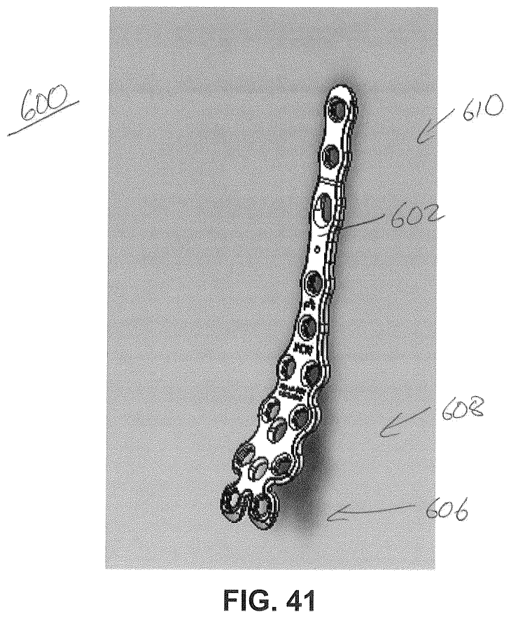

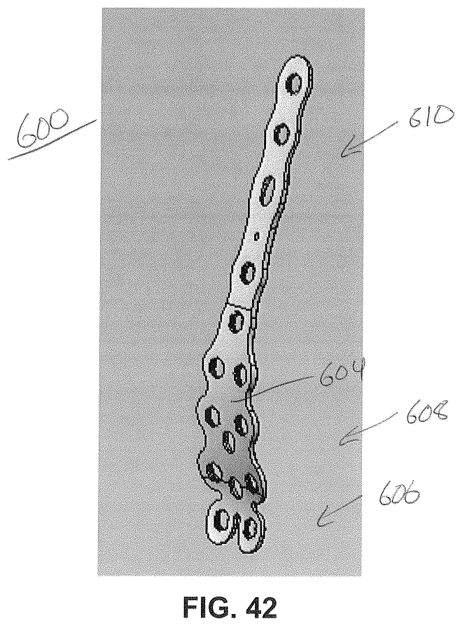

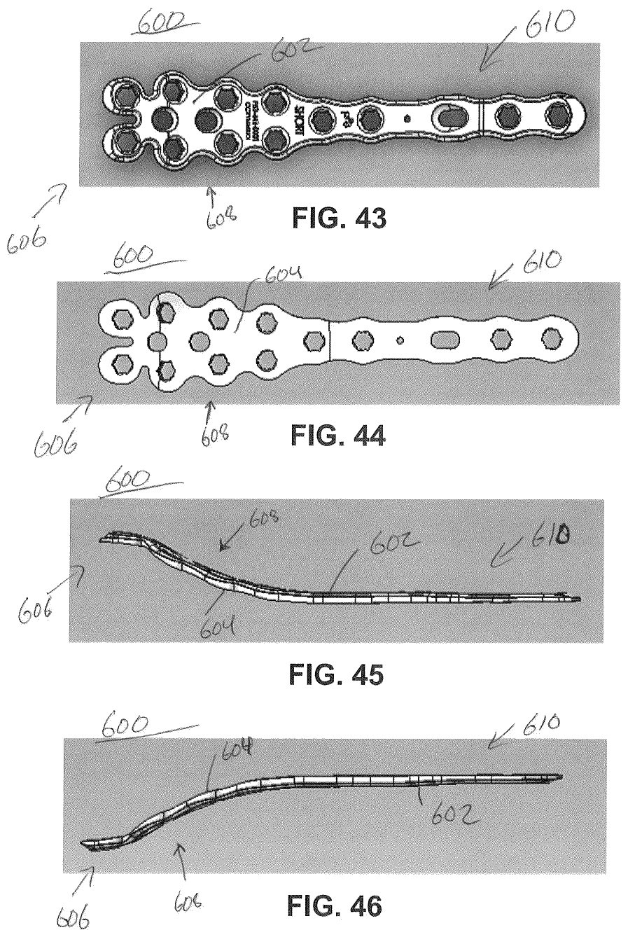

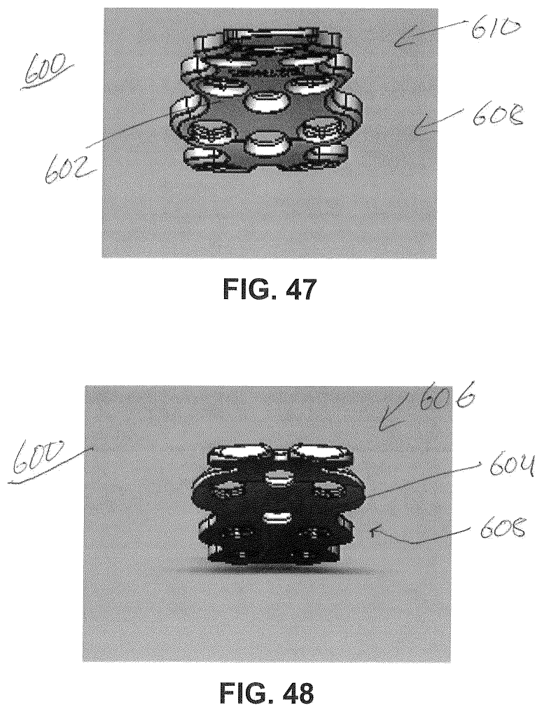

As may be recognized by those of ordinary skill in the art based on the teachings herein, numerous changes and modifications may be made to the above-described and other embodiments of the present disclosure without departing from the scope of the disclosure. The components of the implants as disclosed in the specification, including the accompanying abstract and drawings, may be replaced by alternative component(s) or feature(s), such as those disclosed in another embodiment, which serve the same, equivalent or similar purpose as known by those skilled in the art to achieve the same, equivalent or similar results by such alternative component(s) or feature(s) to provide a similar function for the intended purpose. In addition, the plates may include more or fewer components or features than the embodiments as described and illustrated herein. For example, the features of FIGS. 1-48 may be used interchangeably and in alternative combinations as would be modified or altered by one of skill in the art. Specifically, plates 100, 200, 300, 400, 500 and 600 may be used in alternative combinations as would be modified or altered by one of skill in the art. Accordingly, this detailed description of the currently-preferred embodiments is to be taken in an illustrative, as opposed to limiting of the disclosure.

The disclosure has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the disclosure be construed as including all such modifications and alterations.

What is claimed is:

1. An orthopedic plate, comprising:
an upper surface positioned opposite the plate from a lower surface;
at least one through hole extending between the upper and lower surfaces so as to establish fluid communication therebetween;
a body portion;
a base portion integral with and extending from the body portion; and
an extension integral with and extending from the body portion in a direction substantially opposite that of the base portion.

2. The plate of claim 1, wherein the extension has a lesser width than the body portion or the base portion.

3. The plate of claim 2, wherein the upper surface of the base portion is positioned at a substantially orthogonal angle relative to a front surface of the body portion.

4. The plate of claim 3, wherein the upper surface of the base portion is positioned at an oblique angle relative to at least a portion of the front surface of the extension.

5. The plate of claim 4, wherein the upper surface of the extension is substantially curved.

6. The plate of claim 4, wherein the upper surface of the extension at a terminal end of the extension is positioned in a plane angled at substantially 45-degrees relative to the upper surface of the base portion.

7. The plate of claim 5, wherein the body portion comprises a substantially convex geometry on the upper surface and a substantially concave geometry on the lower surface.

8. The plate of claim 7, wherein the body portion comprises at least one compression slot extending from the upper surface through to the lower surface.

9. An orthopedic plating system, comprising:
an orthopedic plate, comprising:
a body portion;
a base portion integral with the body portion; and
an extension integral with the body portion and the base portion; and
a drill guide.

10. The orthopedic plating system of claim 9, wherein the drill guide is releasably couplable with the body portion of the orthopedic plate.

11. The orthopedic plating system of claim 10, wherein the orthopedic plate comprises upper and lower surfaces substantially opposite from one another and extending along the base portion, the body portion, and the extension.

12. The orthopedic plating system of claim 11, wherein at least a portion of the upper surface has a substantially convex geometry and at least a portion of the lower surface has a substantially concave geometry.

13. The orthopedic plating system of claim 12, wherein the drill guide comprises an upper surface substantially opposite the drill guide from a lower surface, and wherein the upper surface comprises a substantially convex geometry and the lower surface comprises a substantially concave geometry.

14. The orthopedic plating system of claim 13, wherein the body portion comprises:
at least one through hole extending through from the upper surface through to the lower surface; and
at least one coupling feature.

15. The orthopedic plating system of claim 14, wherein the drill guide comprises:
at least one through hole extending from the upper surface through to the lower surface; and
at least one coupling feature having a geometry complementary to that of the coupling feature of the body portion of the orthopedic plate.

16. The orthopedic plating system of claim 15, wherein the at least one through hole of the drill guide is coaxial with the at least one through hole of the body portion of the orthopedic plate when the coupling feature of the drill guide is engaged with the coupling feature of the body portion.

17. The orthopedic plating system of claim 16, wherein the body portion of the orthopedic plate comprises at least two through holes extending from the upper surface through to the lower surface and the drill guide comprises at least two through holes extending from the upper surface through to the lower surface, wherein each of the at least two through holes of the drill guide and the body portion are coaxial when the coupling features of the drill guide and the body portion are engaged with one another.

18. The orthopedic plating system of claim 17, wherein the axes of the coaxial through holes of the drill guide and the body portion form a substantially oblique angle with one another.

19. The orthopedic plating system of claim 17, wherein the axes of the coaxial through holes of the drill guide and the body portion are substantially parallel to one another.

20. The orthopedic plating system of claim 19, wherein the coupling feature of the body portion comprises at least one of a through hole or a depression, and the coupling feature of the drill guide comprises a protrusion.
